(19) United States
(12) Patent Application Publication
(10) Pub. No.: US 2021/0162088 A1
(43) Pub. Date: Jun. 3, 2021

(54) INTELLIGENT SCENT DIFFUSER

(71) Applicant: SMP GMBH, München (DE)

(72) Inventors: Roman BUZNYKOVATYY, München (DE); Guilherme Petterle Silveira MUSSI, Gräfelfing (DE)

(73) Assignee: SMP GMBH, München (DE)

(21) Appl. No.: 17/267,242

(22) PCT Filed: Jul. 15, 2019
(86) PCT No.: PCT/EP2019/069017

§ 371 Date: Feb. 9, 2021

(30) Foreign Application Priority Data
Aug. 14, 2018 (DE) 10 2018 213 686.6

(51) Int. Cl. A61L 9/12; A61L 9/14

(52) U.S. Cl. CPC A61L 9/125; A61L 9/14; A61L 2209/11; A61L 2209/132; A61L 2209/133; A61L 9/122

(57) ABSTRACT

The present invention relates to a scent diffuser for dispensing adaptable scents, comprising a plurality of reservoirs (30) for storing aromatic liquids, a plurality of atomizers (40) for dispersing the aromatic liquids stored in the reservoirs (30) to the outside of the scent diffusor in a controlled manner, wherein to each of the reservoirs (30) at least one atomizer (40) is associated, a control unit (26) operatively coupled to the plurality of atomizers (40), wherein the control unit is adapted to control the operation of the atomizers (40) in a coordinated manner, and a power supply (18) for powering the control unit (26) and the plurality of atomizers (40). The invention further relates to a cartridge containing an aromatic liquid to be used with such a scent diffuser, as well as a method for operating such a scent diffuser.

The present invention relates to a scent diffuser for dispensing adaptable scents, a cartridge containing an aromatic liquid to be used with such a scent diffuser, as well as a method for operating such a scent diffuser.

It has long been known to dispense aromatic liquids such as perfumes or air refresher liquids in the context of cosmetics and other lifestyle fields. For this purpose, manually operated dispensers and diffusers for aromatic liquids are known, with which a user can spray or atomize the liquids upon manual actuation. Additionally, there are electrically operated scent diffusers on the market in which atomizers dispense aromatic liquids for example at regular intervals.

The aromatic liquids to be dispensed are usually complex mixtures of up to dozens or hundreds of aromatic molecules dissolved in carrier or solvent liquids which are mixed in predetermined ratios in order to construct so-called “accords”, a reference to music theory where overlaying multiple tonal frequencies with pre-determined ratios can lead to different sensations of harmony or tension. Based on the concept of said accords, perfume makers classify their perfumes according to family, sub-family and classifier accords, wherein the most intense olfactory sensations are identified as the family, less intense ones are identified as sub-families and the weakest are identified as classifier accords. In the field of perfume composition, said accords are themselves comprised of a number of aromatic molecules and usually categorized according to their real-life origin or archetype, namely into categories such as citric, leathery, woody, etc.

It is apparent that due to the huge number of aromatic liquids found in nature and also commercially available to perfume makers a practically infinite number of possibilities for combining said aromatics is conceivable. Even though this has led to an ever increasing number of perfumes and air refreshers with distinct scents on the market, there is still a demand for personalization of scents. This has for example led to perfume makers offering bespoke scents tailored to their clients who are willing to pay five digit numbers to have their own personalized perfume created. However, all of the scents and perfumes currently on the market are static in such that the mixture ratios of the individual aromatic molecules are fixed by a perfume maker and cannot be dynamically altered and adjusted according to the real-time preferences of the user. Since the human olfactory sense tends to get accustomed to scents rather quickly and due to the static nature of all available perfumes, there occurs the phenomenon of “olfactory fatigue” according to which perfumes and air refreshers tend to subjectively wear off even quicker than the aromatic molecules evaporate.

It is therefore the object of the present invention to provide a scent diffuser for dispensing adaptable scents, which can be personalized to any individual user and adjusted in real time during the operation of the scent diffuser and which can avoid olfactory fatigue. For this purpose, the scent diffuser for dispensing adaptable scents according to the invention comprises a plurality of reservoirs for storing aromatic liquids, a plurality of atomizers for dispersing the aromatic liquids stored in the reservoirs to the outside of the scent diffuser in a controlled manner, wherein to each of the reservoirs at least one atomizer is associated, a control unit operatively coupled to the plurality of atomizers, wherein the control unit is adapted to control the operation of the atomizers in a coordinated manner, and a power supply for powering the control unit and the plurality of atomizers.

By providing a plurality of reservoirs each storing a distinct aromatic liquid and an array of atomizers individually controlled by a common control unit for dispersing the aromatic liquids stored in the reservoirs, not only can an ever increasing number of combined scents be dispersed by the scent diffuser depending on the number of reservoirs employed, but also the scent can be adjusted and altered in real time according to any desired input variable. In this context, the term “controlling the operation of the atomizers in a coordinated manner” relates to the amount of the individual aromatic liquids which is dispersed per time unit or in other words the mass flow rate of each individual aromatic liquid which directly results in the ratio of the aromatic liquids and thus the overall composition of the mixed dispersed scent.

While the number of reservoirs could be as low as two, it is preferable to provide the scent diffuser according to the invention with at least five reservoirs, wherein preferably at least two of them are arranged to store different amounts of aromatic liquid. It is well known that the human olfactory sense has different perception thresholds for different aromatic molecules, such that the concentration of said molecules in the ambient air has to be different to evoke similar sensory stimulations with a particular user of the scent diffuser of the present invention. Even though this phenomenon could be handled by providing the different aromatic liquids in the individual reservoirs at different relative concentrations, depending on the form factor and in order to miniaturize the scent diffuser it can be beneficial to use aromatic liquids with the maximum possible concentration of aromatic molecules and consequently minimize the dispensing rate per time unit of said highly concentrated liquids as well as the volume of the reservoirs containing the liquids.

Again, depending on the form factor and dedicated use case of the scent diffuser according to the invention, its power supply may comprise a rechargeable battery and/or a mains adaptor. In the case a rechargeable battery is provided, a dedicated external power supply such as a charging dock may be provided together with the scent diffuser as well. Concerning the type of rechargeable battery, again the form factor of the scent diffuser has to be taken into account, such that for especially compact devices, lithium ion or lithium polymer batteries with a high energy density might be preferable despite their somewhat higher costs.

In order to guarantee a constant supply of the aromatic liquids stored in the reservoirs to the atomizers, at least one of the reservoirs may contain a capillary sponge which in the loaded state of the reservoir is soaked with the respective aromatic liquid and which guarantees a constant flow of the liquid to the atomizer due to the capillary effect when the respective reservoir is starting to empty. In alternative embodiments, the atomizers might also be positioned below the reservoirs such that the flow of the aromatic liquids to the atomizers might be driven by gravity.

Depending on the projected use case of the scent diffuser according to the invention and thus also on its outer shape, the reservoirs may be replaceable and/or refillable. Especially for very small scent diffusers replaceable reservoirs might be beneficial, since together with the overall device, individual reservoirs also will be quite small and thus difficult to handle in a refilling operation. For bigger units, the reservoirs might be refillable while still positioned in their operational position in the scent diffuser or they might be extracted from it and refilled manually or in a dedicated refilling unit.

In order to further modularize the scent diffuser according to the invention, the atomizers may be integrated with the reservoirs to form replaceable assemblies. Such replaceable assemblies again can be especially beneficial in cases of very small scent diffusers, while in bigger units bigger reservoirs and bigger atomizers can be used which are easier to handle and disassemble for users of the device.

In order to be able to have a feedback on the amount and concentration of the aromatic liquids dispersed to the environment of the scent diffuser, it may further comprise a detection unit for detecting the concentration of at least one type of substance in the vicinity of the diffuser. Thus, the device can automatically detect errors in the dispersing process or can adjust the mass flow rate of the aromatic liquids based on the detection results, such that for example for an air refresher unit arranged in an apartment or a car in cases in which due to a draught the dispersed aromatic liquids are quickly distributed in the room to be refreshed, the mass flow rate might be increased accordingly.

While the control unit of the scent diffuser according to the invention might be provided with fixed profiles based on which its operation is controlled concerning dispersing of the aromatic liquids and their relative mass flow ratios, the diffuser may further comprise an input device for receiving instructions concerning the operation of the scent diffuser, communicatively coupled to the control unit, in particular comprising a communication unit for receiving instructions from an external device. Thus, by providing the scent diffuser with appropriate interfaces, such as a Bluetooth-transceiver, the user of the scent diffuser according to the invention may adjust parameters of the operation of the scent diffuser based on input data from an external Bluetooth-enabled device such as his/her mobile phone. In other embodiments, different wireless communication standards such as WIFI or ZigBee might be used and also a wire-based information exchange is conceivable after plugging in an external device in a dedicated port of the diffuser. In other embodiments, the scent diffuser itself might be provided with a user interface such as a touchscreen and/or hard buttons.

While the atomizers employed in the scent diffuser according to the invention can be of any type which is able to disperse liquids with a suitable mass flow rate and precision, in a preferred embodiment at least one of the atomizers may comprise a micro-perforated membrane and an ultrasonic transducer for oscillating the membrane, preferably a piezo-effect transducer. Such atomizers are durable and have shown to be especially suitable for applications in which small amounts of liquids have to be dispersed with high precision.

In one embodiment, a scent diffuser according to the invention may further comprise a diffuser housing which fully encloses at least the reservoirs, and which is provided with at least one outlet hole through which the dispersing of the aromatic liquids can take place, wherein the housing is preferably shaped and dimensioned as a stand-alone unit or for integration into a higher level structure such as a car. By providing at least the reservoir and preferably also the control unit and the power supply and possible additional components inside a housing, the scent diffuser can become sufficiently sturdy for many applications and use cases, and by choosing the outer shape of the housing accordingly, it can be integrated in any kind of receptacle such as the cup holder of a car.

In order to guarantee a satisfactory distribution of the dispersed aromatic liquids, the scent diffuser may further comprise at least one inlet hole and a ventilation device for causing an air flow from the at least one inlet hole through the housing to the at least one outlet hole, such that the dispersed aromatic liquids are carried outside the housing and further distributed in the environment of the scent diffuser by a constant or variable flow of air.

In order to further enhance the sensory experience of the user of the scent diffuser and to provide optical feedback on its operational status, the housing may be made from a transparent material, and preferably inside the housing at least one LED may be provided which is powered by the power supply and controlled by the control unit. Thus, the dispersion of the aromatic liquids and the operation of the LEDs may be controlled in a coordinated manner, such that for example depending on the current mixture of aromatic liquids and thus the overall scent dispersed at a certain time, the color and/or intensity of the LEDs might be adjusted.

According to another embodiment of the scent diffuser according to the invention, it may comprise a band, in particular a wrist band, which is adapted to be worn by a user, wherein the reservoirs are individually embedded in slots formed in the band. By providing the scent diffuser with the wearable band, the device becomes a mobile wearable unit which is capable of periodically renewing a perfume application to the part of the body where the user wears it, in particular his or her wrist. It is common to apply perfumes to the wrist, since due to the high blood circulation close to the skin of the wrists, the aromatic molecules of the perfume will be evaporated efficiently. However, due to the evaporation, a frequent re-application of the perfume to the wrist might be necessary. The described embodiment of the scent diffuser according to the invention is capable of doing exactly that, namely periodically refreshing the user's perfume application on his or her wrist. By embedding the reservoirs individually in slots formed in the band, the overall size of the scent diffuser can be reduced and the flexibility of the band can be preserved, such that the scent diffuser can be comfortably worn over long periods of time.

In order to have the aromatic liquids dispersed towards the skin of the user, at least one of the atomizers, preferably all of the atomizers, can be arranged towards the inner circumference of the band. However, if it is not desired to exclusively bring the aromatic liquids in direct contact with the skin of the wearer of the band, at least one of the atomizers may of course also point to the outside of the band, such that the aromatic liquids are dispersed to the environment of the band and its wearer.

The just described scent diffuser comprising a band may further comprise a display device, preferably a touchscreen, operatively coupled to the control unit. Thus, the device may serve many additional purposes, such as having smart watch functionalities or the touchscreen may serve as the input device of the scent diffuser.

According to a second aspect of the present invention, a cartridge containing an aromatic liquid and provided with at least one atomizer for dispersing said aromatic liquid to the environment and an interface for receiving power for operating the atomizer is provided, wherein the cartridge is shaped and dimensioned to serve as a reservoir of a scent diffuser comprising a band to be worn by a user as discussed above. Such cartridges integrated with atomizers and able to receive power to operate them can be provided as interchangeable units and easily replaced by the user of the scent diffuser once empty or to be replaced by a different scent. The cartridges may further be provided with a robust shell, since depending on the shape of the band and the slots formed therein for accommodating the cartridges, the shell may be exposed to the outside due to the cartridge not being fully enclosed by a housing or case.

Furthermore, the present invention relates to a method of operating a scent diffuser according to the invention, wherein the control unit operates the atomizers such that the mass flow per time unit of the aromatic liquids dispersed by the individual atomizers is controlled to a desired ratio. Herein, the desired ratio may be set or adjusted according to user input, recommendations or according to principles derived with artificial intelligence based on all sorts of input variables.

In order to avoid the above-discussed olfactory fatigue, the ratios of the mass flows per time unit of the dispersed aromatic liquids may be time-modulated in a predetermined or random manner with predefined boundaries. By time-modulating the composition of the overall scent dispersed by the scent diffuser, depending on the predefined boundaries, the “tonality” of the resulting overall scent may be preserved or altered to a desired degree, while the total intensity of the scent may also be altered to a degree which is perceivable by the user or not, depending on the desired mode of operation.

While the dispersing of the aromatic liquids may be performed continuously, in order to safe resources such as the aromatic liquids stored in the reservoirs, power and lifetime of the atomizers, it may be beneficial to perform the dispersing of the aromatic liquids intermittently according to predetermined intervals. Said intervals can of course be set to fixed values or adjusted according to different parameters such as time of day, detection results of a sensor unit such as described above, user input or similar.

Lastly, the method according to the invention may comprise a detection of the concentration of at least one type of substance in the vicinity of the diffuser and adjusting the mass flow per time unit of at least one of the dispersed aromatic liquids based on said detection. While depending on the chemical compounds used as aromatic molecules and the availability of sensors for detecting said molecules any compound used in the aromatic liquids provided in the reservoirs might be employed for detecting its concentration, it can be especially beneficial to add one or more non-smelling and non-harmful chemical compounds as a chemical marker to the aromatic liquids provided in the reservoirs in order to be detected as a measure for the mass flow rate from the respective reservoir.

Additional features and benefits of the present invention will become even clearer from the following description of embodiments of the present invention when considered together with the attached figures. These figures show:

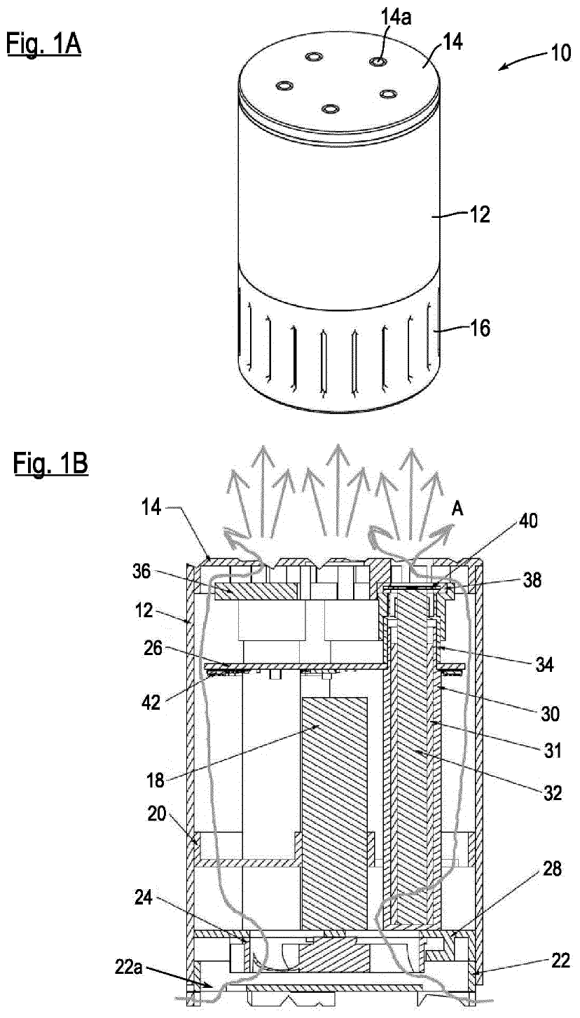

FIGS. 1A and 1B a stand-alone embodiment of a scent diffuser according to the present invention in perspective and in cross-sectional views;

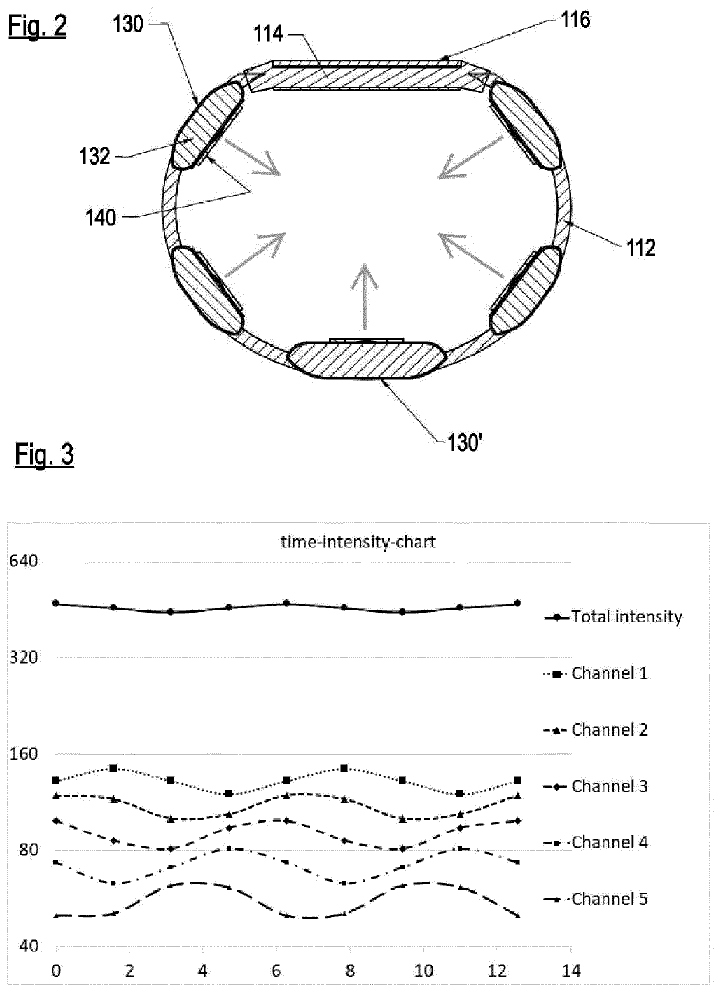

FIG. 2 an alternative embodiment of a scent diffuser according to the present invention in cross-sectional view; and

FIG. 3 a time-intensity chart of the mass flow rate of the aromatic liquids as dispensed by the embodiments of FIGS. 1A to 2.

In FIG. 1A, an embodiment of a scent diffuser according to the invention is shown and generally denoted with reference numeral 10. The scent diffuser 10 comprises a cylindrical housing 12 with a top cover 14 and can serve as a stand-alone unit, for example as an air refresher for apartments. The housing 12 could have various other shapes, for example could be cup-shaped to be placed into cup holders in cars.

In the top cover 14, five outlet holes 14a are provided through which during an operation of the scent diffuser multiple aromatic liquids are dispersed to the outside of the scent diffuser 10. Furthermore, in FIG. 1A, a charging station 16 is shown, onto which the scent diffuser 10 is currently positioned in order to charge its battery pack 18, which is shown in the sectional view of FIG. 1B.

Said rechargeable battery 18 is held in place by a battery holder 20 which is fixed to the casing 12 and provided with suitable through holes for holding the battery and for allowing an air flow inside the housing 12. In order to cause said air flow, in the bottom plate 22 of the scent diffuser 10, multiple inlet holes 22a are provided and an air flow entering the housing 12 through the inlet holes 22a is caused by the operation of a low-noise ventilation fan 24 powered by the battery 18 and controlled by a control device 26 which is provided in form of a circuit board above the battery 18 inside the housing 12.

The ventilation fan 24 is mounted to a lower holder 28 positioned in the lower part of the housing 12 and on which the battery 18 also rests. Said lower holder 28 is also provided with through holes for allowing the above-mentioned air flow through the housing 12 from the inlet holes 22a to the outlet holes 14a. Also positioned on the lower holder 28 are five reservoirs 30 of which in the sectional view of FIG. 1B only three are shown. The reservoirs 30 are provided in the form of glass vials each containing a capillary sponge 32 which is soaked with an aromatic liquid 31. In the upper part of the reservoirs 30, the glass vials are provided with an outer thread such that they can be screwed into respective holders 34 with counter-threads. In the embodiment of FIGS. 1A and 1B, the bottom plate 22 is removable from the housing 12 in order to give the user access to the interior of the housing 12 for replacing the reservoirs 30.

Further on top of the reservoir holders 34, an upper holder 36 is provided which holds the reservoirs from the above and which is provided with membrane holders 38 in respective passages above the individual reservoirs 30. The membrane holders 38 are made of soft rubber and hold micro-perforated membranes 40 which serve as ultrasonic transducers for dispersing the aromatic liquids 31 contained the reservoirs 30. Said aromatic liquids 31 are continuously drawn in the direction of the membranes 40 by the capillary effect of the capillary sponge 32 provided inside the reservoirs 30 during operation of the membranes 40.

For this purpose, the membranes 40 are piezo-driven and powered by voltages supplied from the battery 18 and controlled by the control device 26 for dispersing small amounts of the aromatic liquids contained in the reservoirs 30 per time unit continuously or intermittently. The voltage supplied by the battery 18 may further be converted by a step-up converter (not shown) depending on the type of membrane 40 employed, for example up to about 50V. The liquids are atomized towards the top cover 14 inside the housing 12 and dispensed through the outlet holes 14a by the air flow caused by the ventilation fan 24 as indicated by the arrows A.

By controlling the operation of the five membranes 40 individually and in a time-dependent manner, the control device 26 can adjust the ratios of the mass flows of the aromatic liquids contained in the five reservoirs 30 in order to create the desired scent in the environment of the scent diffuser 10 as a mixture of the individual scent accords provided by the five reservoirs 30.

In order to enhance the sensory experience of the user of the scent diffuser 10, the housing 12 is made of a translucent plastic material and a number of LEDs 42 are provided to the printed circuit board and controlled by the control unit 26 in unison with the operation of the membranes 40. Furthermore, the control unit 26 is provided with a wireless communication module, such as a Bluetooth module, for wireless communication with an external device such as a remote control or a mobile phone running a dedicated software application for controlling the operation of the scent diffuser 10, with which all parameters of the operation of the scent diffusor 10 can be set and adjusted, such as composition of the scent, mass flow rates of the dispersion of the individual aromatic liquids, start and stop times, intervals for the operation of the device and so on.

FIG. 2 shows an alternative embodiment of a scent diffuser according to the invention, generally denoted with the reference numeral 100. Said scent diffuser 100 is provided with a band 112 which serves as a wrist band such that the scent diffuser 100 of FIG. 2 can be worn similarly to a bracelet or a watch. The basic operational principles of the embodiment of FIG. 2 are similar to the embodiment of FIGS. 1A and 1B, such that in the following description, mostly the differences between the embodiments will be pointed out and for all additional aspects it is referred to the above description of the first embodiment.

The scent diffuser 100 furthermore comprises a main panel 114, in which a touchscreen 116 is integrated which serves as an input device for the operation of the scent diffuser 100 and which can furthermore perform additional tasks generally known from available smart watches or similar devices. The main panel 114 also houses further components such as the control unit, the communication module and the battery of the scent diffuser 100 which are not shown in the view of FIG. 2.

In contrast to the scent diffuser 10 of FIGS. 1A and 1B, in the embodiment according to FIG. 2, the reservoirs containing the aromatic liquids are not enclosed in a housing, but are rather embedded in slots 112a formed in the band 112 and fixed thereto by a releasable snap-fit connection. The reservoirs 130 are furthermore directly connected to the membranes 140, such that they constitute a non-separable cartridge which can be fitted into the band 112 and removed therefrom in one piece. Through the snap fit of the reservoirs 130 into the band 112 connections for powering the membranes 140 are made and corresponding wires are integrated into the band 112 such that the control unit integrated in the main panel 114 can control the individual membranes 140 by applying voltages to them.

Since the cartridges are partially exposed to the outside, they are provided with a robust outer shell which again houses a capillary sponge 132 soaked with a respective aromatic liquid. It can be further seen that the reservoirs 130 are not all of the same size, in particular the reservoir 130′ opposed to the main panel 140 is of a larger dimension and thus can hold a bigger volume of aromatic liquid. Since the membranes 140 are oriented towards the inner circumference of the band 112, they will disperse the aromatic liquid contained in the respective reservoir 130 towards the wrist of its wearer where the individual aromatic liquids will collectively serve as his or her perfume.

It is a well-known fact that the human olfactory sense tends to quickly get accustomed to scents, a phenomenon commonly referred to as “olfactory fatigue”. In order to avoid olfactory fatigue during the operation of scent diffusers according to the invention such as shown in FIGS. 1A to 2, the operation of the individual membranes for dispersing the aromatic liquids may be performed as shown in FIG. 3, in which a time-intensity chart is shown, with the time in minutes represented on the X-axis and the relative mass flow rate of the five aromatic liquids dispersed by the membranes 40, 140, respectively, on the Y-axis. Herein, the five different aromatic liquids are all dispersed with a respective mass flow rate, which adds up to a total intensity. Each of the mass flow rates of the five aromatic liquids is varied within appropriately a 20% deviation window over time, in a manner that the overall intensity varies only within appropriately a 6% window and also in a manner that the relative ordering of the mass flow rates stays the same over time, i.e. the flow rate lines do not cross at any time. By operating the five membranes in such a manner, the olfactory sense of the user will not grow tired of the scent over time, however, the overall intensity as well as the general composition of the combined scent of the five aromatic liquids will hardly change for his or her perception.

1. Scent diffuser for dispensing adaptable scents, comprising:
a plurality of reservoirs for storing aromatic liquids;
a plurality of atomizers for dispersing the aromatic liquids stored in the reservoirs to the outside of the scent diffusor in a controlled manner, wherein to each of the reservoirs at least one atomizer is associated;
a control unit operatively coupled to the plurality of atomizers, wherein the control unit is adapted to control the operation of the atomizers in a coordinated manner; and
a power supply for powering the control unit and the plurality of atomizers.

2. Scent diffuser according to claim 1, wherein at least five reservoirs are provided, wherein at least two of the reservoirs are arranged to store different amounts of aromatic liquid.

3. Scent diffuser according to claim 1, wherein the power supply comprises a rechargeable battery and/or a mains adaptor.

4. Scent diffusor according to claim 1, wherein at least one of the reservoirs contains a capillary sponge.

5. Scent diffusor according to claim 1, wherein the reservoirs are replaceable and/or refillable.

6. Scent diffusor according to claim 1, wherein the atomizers are integrated with the reservoirs to form replaceable assemblies.

7. Scent diffusor according to claim 1, further comprising a detection unit for detecting the concentration of at least one type of substance in the vicinity of the diffusor.

8. Scent diffusor according to claim 1, further comprising an input device for receiving instructions concerning the operation of the scent diffusor, communicatively coupled to the control unit, wherein the input device comprises a communication unit for receiving instructions from an external device.

9. Scent diffusor according to claim 1, wherein at least one of the atomizers comprises a micro-perforated membrane and an ultrasonic transducer for oscillating the membrane.

10. Scent diffusor according to claim 1, further comprising a diffusor housing, which fully encloses at least the reservoirs and which is provided with at least one outlet hole through which the dispersing of the aromatic liquids takes place, wherein the housing is shaped and dimensioned as a stand-alone unit or for integration into a higher-level structure such as a car.

11. Scent diffusor according to claim 10, further comprising at least one inlet hole and a ventilation device for causing an airflow from the at least one inlet hole through the housing to the at least one outlet hole.

12. Scent diffusor according to claim 10, wherein the housing is made from a transparent material, wherein inside the housing at least one LED is provided which is powered by the power supply and controlled by the control unit.

13. Scent diffusor according to claim 1, further comprising a band, which is adapted to be worn by a user, wherein the reservoirs are individually embedded in slots formed in the band.

14. Scent diffusor according to claim 13, wherein at least one of the atomizers is arranged towards the inner circumference of the band.

15. Scent diffusor according to claim 13, further comprising a display device operatively coupled with the control unit.

16. Cartridge containing an aromatic liquid and provided with at least one atomizer for dispersing said aromatic liquid to the environment and an interface for receiving power for operating the atomizer, wherein the cartridge is shaped and dimensioned to be serve as the reservoir of a scent diffusor according to claim 13.

17. Method for operating a scent diffusor according to claim 1, wherein the control unit operates the atomizers such that the mass flow per time unit of the aromatic liquids dispersed by the individual atomizers is controlled to desired ratios.

18. Method according to claim 17, wherein the ratios of the mass flows per time unit of the dispersed aromatic liquids is time-modulated in a pre-determined or random manner within pre-defined boundaries.

19. Method according to claim 17, wherein the dispersing of the aromatic liquids is performed intermittently according to pre-determined intervals.

20. Method according to claim 17, further comprising detecting the concentration of at least one type of substance in the vicinity of the diffusor and adjusting the mass flow per time unit of at least one of the dispersed aromatic liquids based on said detection.
